(19) United States
(12) Patent Application Publication
(10) Pub. No.: US 2014/0277203 A1
(43) Pub. Date: Sep. 18, 2014

(54) TORQUE MULTIPLIER, LIMITER, AND COUNTER-TORQUE COMBINATIONS AND METHODS

(71) Applicant: EBI, LLC, Parsippany, NJ (US)

(72) Inventors: Matthew ATOULIKIAN, Parsippany, NJ (US); Oliver BUCHERT, Franklin Lakes, NJ (US); Daryl CARTER, Hapatcong, NJ (US); Elwood H. GREENE, Allentown, PA (US); Roman Marion STECIUK, Towaco, NJ (US)

(73) Assignee: EBI, LLC, Parsippany, NJ (US)

(21) Appl. No.: 14/106,170

(22) Filed: Dec. 13, 2013

Related U.S. Application Data
(60) Provisional application No. 61/783,096, filed on Mar. 14, 2013.

(51) Int. Cl. A61B 17/70

(52) U.S. Cl. CPC A61B 17/7091
USPC 606/86 A

(57) ABSTRACT

A drive train includes a torque multiplier assembly and a torque limiter device. An output of the torque multiplying assembly is drivingly coupled to an input of the torque limiter device. An input of the drive train is configured to be driven by a driver handle and an output of the drive train is configured to rotate a driver. A counter-torque connector that is configured to engage a spinal fixation construct is coupled to a counter-torque handle to prevent rotation of the spinal fixation construct. A coupling between a component of the torque multiplier assembly and the counter-torque handle prevents rotation of the component. Torque input by the single user through the driving handle is simultaneously multiplied by the torque multiplier assembly and limited by the torque limiter device, while the single user simultaneously prevents the spinal fixation construct from rotating through the counter-torque handle.

### CROSS-REFERENCE TO RELATED APPLICATIONS

This application claims the benefit of U.S. Provisional Application No. 61/783,096, filed on Mar. 14, 2013. The entire disclosure of the above application is incorporated herein by reference.

### BACKGROUND

This section provides background information related to the present disclosure which is not necessarily prior art.

In some applications it is necessary to hold a driver in one hand to apply a torque to a fastener or plug, while holding a counter-torque device in the other hand to prevent a surrounding member from rotating. This occurs, for example, during spinal fixation surgery where a spinal construct includes a rod extending through an end of a fastener where the rod is locked in place by a plug that must be torqued to a predetermined and significant degree relative to a surrounding fastener. For example, 110 in-lbs or more of torque can be required, and a torque limiter can be provided to insure the proper amount torque is applied.

It is difficult for a surgeon to apply the required significant torque/counter-torque during surgery. Unfortunately, torque multiplying devices typically require the user to hold the housing of the torque multiplier, but both hands are already occupied as discussed above.

In addition, torque limiters typically require some axial movement of the input shaft when the torque limit is reached. This provides an additional barrier to the use of a torque multiplying device.

### SUMMARY

This section provides a general summary of the disclosure, and is not a comprehensive disclosure of its full scope or all of its features.

A method of providing a torque multiplier, limiter, and counter-torque combination for use in spinal fixation surgery includes providing a drive train comprising a torque multiplier assembly and a torque limiter device. An output of the torque multiplying assembly is drivingly coupled to an input of the torque limiter device. An input of the drive train is configured to be driven by a driver handle and an output of the drive train to rotate a driver. A counter-torque connector that is configured to engage at least one member of a spinal fixation construct is coupled with a counter-torque handle to prevent rotation of the counter torque coupling and the spinal fixation construct. A coupling is provided between a component of the torque multiplier assembly and the counter-torque handle to prevent a rotation of the component. The driver handle and counter-torque handle are configured to be simultaneously engaged by a single user, wherein torque input by the single user through the driving handle is simultaneously multiplied by the torque multiplier assembly and limited by the torque limiter device, while the single user simultaneously prevents the at least one member of the spinal fixation construct from rotating through the counter-torque handle.

A torque multiplier, limiter, and counter-torque combination for use in spinal fixation surgery includes a drive train comprising a torque multiplier assembly and a torque limiter device. An output of the torque multiplying assembly is drivingly coupled to an input of the torque limiter device. An input of the drive train is coupled to a drive handle and an output of the drive train is coupled to a driver. A counter-torque device comprises a counter-torque connector configured to engage at least one member of a spinal fixation construct and a counter-torque handle to prevent rotation of the counter torque coupling and the spinal fixation construct. A component of the torque multiplier assembly is coupled to the counter-torque handle to prevent a rotation of the component. A torque input through the driving handle is simultaneously multiplied by the torque multiplier assembly and limited by the torque limiter device at the driver. A counter-torque simultaneously input through the counter-torque handle simultaneously prevents rotation of the component of the torque multiplier assembly and rotation of the at least one member of the spinal fixation construct.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a perspective view of one example of a torque multiplying driver and limiter configured in accordance with the present disclosure.

FIG. 2 is a similar perspective view of the torque multiplying driver of FIG. 1 with the outer housing removed.

FIG. 3 is a slight perspective view of the torque multiplying driver of FIG. 1 with an output driver coupled thereto;

FIG. 4 is a perspective view similar to FIG. 3 with a counter-torque device keyed to the torque multiplying driver of FIG. 1.

FIG. 5 is an enlarged partial perspective view of the torque multiplying driver and limiter of FIG. 1 keyed to the counter-torque device.

FIG. 6 is a perspective view of the counter-torque device alone.

FIG. 7 is a slight perspective view of the torque multiplying driver limiter and counter-torque device of FIG. 4, positioned on a rod and screw, which has been enlarged.

FIG. 7A is an enlarged perspective view of an exemplary screw and plug assembly as illustrated in U.S. Pat. No. 7,699,876, which is hereby incorporated herein in its entirety.

FIG. 8 is a perspective view of the torque multiplying driver of FIG. 1 with the outer housing removed.

FIG. 9 is a side elevation view of the torque multiplying driver of FIG. 1 with the outer housing removed.

FIG. 10 is a side elevation view of the torque multiplying driver of FIG. 1 including the outer housing.

FIG. 11 is a cross-sectional view of the torque multiplying driver and limiter taken along line 11-11 of FIG. 10.

FIG. 12 is a cross-sectional view of the torque multiplying and driver and limiter taken along line 12-12 of FIG. 10.

FIG. 13 is a side elevation view of the torque multiplying driver and limiter of FIG. 1 with the outer housing removed and including an enlarged view of the gear assembly.

FIG. 14 is a cross sectional view of another example of a torque multiplying driver combination configured in accordance with the present disclosure.

FIG. 15 is an enlarged partial cross-sectional view of the torque multiplying driver combination of FIG. 14.

FIG. 16 is a representative spinal construct similar to FIG. 7A.

FIG. 17 is an enlarged cross sectional view taken along line 17-17 of FIG. 14 in combination with a top plan view of the construct of FIG. 16.

FIG. 18 is an enlarged partial cross-sectional view of a slip coupling of the torque multiplying driver combination of FIG. 14.

FIG. 19 is an enlarged partial cross-sectional view of the torque multiplying driver combination similar to FIG. 15 identifying various dimensions.

Corresponding reference numerals indicate corresponding parts throughout the several views of the drawings.

### DETAILED DESCRIPTION

Example embodiments will now be described more fully with reference to the accompanying drawings. Although the torque multiplier, limiter and counter-torque device combination and methods are described and illustrated herein in the context of surgical procedures, including spinal fixation constructs, those of ordinary skill in the art will appreciate that the screw driver disclosed herein, in its broadest aspects, has further applicability.

One example of a torque multiplying driver and limiter 20, and such a driver 20 in combination with a counter-torque device 22 is illustrated in FIGS. 1-13. Torque multiplying driver and limiter 20 includes an outer housing 66, and an input shaft 26 drivingly coupled to an output 28 via a torque multiplying assembly 30 and a torque limiter 32. Input shaft 26 can have a standard ¼ inch square quick connect geometry that can be attached to standard medical device handles that utilize compatible female geometries. One of ordinary skill will appreciate that other coupling configurations are possible.

An input gear 34 is mounted on input shaft 26 to rotate with input shaft 26. Input gear 34 is drivingly engaged with a first set of three relatively larger driven gears 36, each mounted at a proximal end of a shaft 38 to rotate with spline shaft 38. Rotation of the spline shafts 38 rotates a second set of three relatively smaller gears 40 mounted at a distal end of each spline shaft 38 to rotate with the spline shaft 38. An output gear 42 is mounted on an output shaft 44 of the torque multiplying assembly 30 to rotate with the output shaft 44. This torque multiplying assembly 30 can result in a 2:1 torque multiplication between the input shaft 26 and the output shaft 44. One of ordinary skill will appreciate that other torque multiplication ratios are possible.

A fixed coupling 46 joins output shaft 44 to an input shaft 48 of the torque limiter 32 such that shafts 44 and 48 rotate together. Medical grade torque limiters are commercially available from Beere Medical of Kenosha, Wis. For example, the internal components of torque limiter 32 can be those of Beere Medical's commercially available Part No. TM201. Torque limiting mechanism 32 limits the magnitude of the output torque to a predetermined amount. For example, the output torque can be limited to about 110 in-lbs in accordance with the Polaris™ 5.5 surgical technique. One of ordinary skill will appreciate that other torque limits are possible.

Torque limiting mechanism 32 includes axial motion of the limiter's input shaft 48 (to the left in FIG. 11) in order to mechanically limit the output torque when the torque limit has been met. Therefore output gear 42 and output shaft 44 of torque multiplying assembly 30, which are coupled to the input shaft 48 of the limiter, have been designed to accommodate a predetermined amount of axial translation of shaft 48.

In this example, gaps 50 and 52 are provided in torque multiplying assembly 30. Specifically, gap 50 is provided between the end of shaft 44 and a coupling plate 54. Similarly, gap 52 is provided between gear 42 and a shoulder of coupling plate 54. Thus, axial movement at the end of shaft 44 is permitted, while the axial position of input shaft 26 is unaffected by this axial movement of shaft 44. Alternatively or additionally, coupling 46 can be configured to allow the end of shaft 48 to move axially (to the left in FIG. 11) without causing axial movement of shaft 44 via gap 56. Thus, an axial slip coupling is provided between input shaft 26 of the driver 20 and of input shaft 48 of torque limiter 32.

Output 28 of torque limiting mechanism 32 can have a standard ¼ inch square quick connect female geometry, to retain a driver 58 that can be used to interface with and tighten the helical flange plugs within the Polaris™ 5.5 Spinal System. One of ordinary skill will appreciate that driver 58 and coupling 28 can be of any appropriate configuration, and that the torque multiplying driver and limiter 20 can be used in other applications. Thus, the drive train of the combination extends from input of shaft 26 to output 28 and includes at least shafts 26, 38, 44, and 48, and gears 34, 36, 40, and 42, and coupling 46.

Torque multiplying driver and limiter 20 can be used with a counter-torque device 22. Counter-torque device 22 includes a handle 60, a first tubular member 62 for receiving driver 58, and a second tubular member 64 for receiving at least part of the outer housing 66 of torque multiplying driver and limiter 20. Counter-torque device 22 is keyed to housing 66 of torque multiplying driver 20. Accordingly, holding handle 60 of counter-torque device 22 to prevent rotation of screw or fastener 72 or rod 74, or both, during final tightening simultaneously operates to prevent outer housing 66 of torque multiplying driver 20 from rotating while the inner mechanism is allowed to rotate. The torque multiplying action can require that the outer housing 66 be kept rotationally stationary in this manner.

In this example, the keyed configuration includes a plurality of elongated protrusions or lug members 68 extending axially along an inner surface of tubular member 64 of counter-torque device 22, which engage into cooperating elongated slots 70 along an outer surface of outer housing 66 of torque multiplying driver 20. As a result, a user is not required to separately hold counter-torque instrument 22 and the torque multiplying driver 20 to prevent its housing 66 from rotating. A single hand on a single handle 60 accomplishes both needs. Thus, lug member 68 engaged in slot 70 provides a coupling to prevent a rotation of a component of torque multiplier assembly 30 (e.g., housing 66, plate 54, spline shafts 38 (orbital rotation)). One of ordinary skill will appreciate that the keyed configuration can be any arrangement that allows counter-torque device 22 to resist rotation of housing 66 of driver 20.

The distal end of the counter-torque device 22 is configured to key with or otherwise engage the head 77 of the fastener 72 or the rod 74, or both to prevent the fastener 72 and rod 74 from rotating as torque is applied to the plug 76. For example, outer peripheral surface of head 77 of fastener 72 can include a plurality of outwardly disposed flats 78 that mate with cooperating inwardly disposed flats of extensions 80 at the distal end of counter-torque device 22. Additionally or alternatively, rod 74 can be engaged in recess 81 for counter-torque purposes. In this example, any of extensions 80, recess 81, or both can provide a counter-torque connector for coupling with the construct.

The user can be alerted through tactile and audible feedback when the appropriate amount of torque has been applied. As an example, the resistance felt at the input shaft 26 can be released and a click can be heard once the required predetermined torque has been achieved. In other examples, no signal audible or tactile signal may be provided, or a visible indication may be provided.

The proposed devices 20 and 22 can be manufactured from standard medical grade materials, such as stainless steel and titanium, and contains openings along the housing members for ease of cleaning and steam penetration. Thus, the above-described combination of a torque multiplier, torque limiter, and a driver can be operated by a surgeon using only two hands. In low torque applications, the above described combination may be capable of one-handed operation, with the keying interaction being capable of providing sufficient counter-torque.

Alternatively or additionally, handle 60 of counter-torque device 22 can be positioned at or near the same generally transverse plane as a driving handle 61 of the driver 20 coupled to shaft 26, and with the couplings of the handles closely radially or angularly positioned to form an angle allowing both handles to be grasped in one hand. In other words, each of the handle of driver 20 and handle 60 of counter-torque device 22 can be positioned in a trigger-type (or “V”-type) handle configuration (similar to that described below, but with the two handles spaced or angled relative to each other along a plane that is generally transverse to the rotating driver 58) to permit one-handed operation of all three components during final tightening (at least).

Another example of a torque multiplying driver combination that can include a counter-torque device 120 is illustrated in FIGS. 14-19. In this example a combination of a torque multiplier assembly 130, torque limiter 132, and counter-torque device 122 can be operated using only one hand. Here the two handles are spaced or angled relative to each other along a plane that is coincident or parallel to the central axis of rotating driver 158. In addition, this combination converts linear motion to rotary motion.

A user squeezes a driving handle member 161 toward second handle member 160, rotating handle member 161 about pivot 159. This advances a driving pawl 182, which in turn drives a rack 184 forward within the housing 166. Rack 184 rides in a slot and is coupled to counter-torque handle 160 via housing 166 to prevent rotation of axially movable rack 184 component or torque multiplier assembly 130, causing barrel 190 to rotate. Driving pawl 182 is pivotably coupled to handle member 161 via pivot 187 and biased in a counter-clockwise direction via biasing member or spring 196. When advancing, at least one tooth 183 of driving pawl 182 engages at least one of the teeth 185 of rack 184.

As rack 184 moves forward, a pin 186 on its top surface rides along a spiral or helix slot 188 of a barrel cam 190. This causes barrel cam 190 to rotate within housing 166. Barrel cam 190 is prevented from moving forward within housing 166 by wall 192. Thus, input torque generated by rotational movement of driving handle 161 is multiplied as it is converted to linear movement of rack 184 and to rotational movement of barrel cam 190 providing a multiplied output torque to torque limiter 132.

Barrel cam 190 is coupled to an input shaft 148 to the in-line torque limiter 132, which limits the torque transmitted to driver 158 to a predetermined value. Thus, rotation of barrel cam 190 rotates input shaft 148 of torque limiter 132, which in turn rotates female coupling 128 and driver 158 mated therein.

A holding pawl 194 holds rack 184 in position between handle member 161 strokes toward handle member 160. A holding pawl 194 is pivotably coupled to housing 166 via pin 197 and is biased by biasing member or torsion spring 195 in a counter-clockwise direction. The resulting engagement between teeth 193 and 185 of holding pawl 194 and rack 184, respectively, prevents rack 184 from sliding back (toward the right in FIG. 14). In this way the rack 184 is driven progressively forward (toward the left in FIG. 14) with each handle member 161 stroke toward handle member 160, which in turn results in driver 158 rotating further with each stroke.

Once plug 176 in fastener or screw 172 contacts rod 174, the torque at the driver 158 increases until the predetermined torque limit of torque limiter 132 is reached. Upon reaching the predetermined torque limit input shaft 148 of torque limiter 132 moves axially back (right in the FIG. 14) towards axial slip joint or coupling 154. As noted above, barrel cam 190 is prevented from moving forward (left in FIG. 14) by internal flange or wall 192 of housing 166. Gap 150 within axial slip joint or coupling 154 allows input shaft 148 of the torque limiter 132 to move axially within coupling 154.

The slot 181 at the distal end of the combination provides a connector for counter-torque 122 for rod 174 in the construct as shown in FIG. 16 via at least handle member 160. As such, the user does not need to grasp a separate counter-torque with a second hand.

Torque multiplying mechanism 130 provides this capability through mechanical advantage. Mechanical advantage is achieved by increasing the distance over which the handle force is applied (via multiple handle squeezes) while limiting the amount of incremental rotation of the barrel cam 190 via the helix angle of barrel cam slots 188.

In addition to the helix angle of barrel cam slots 88, FIG. 19 shows some ratios that can be used to generate the desired mechanical advantage for this embodiment. Increasing the ratio of A to B (handle pivot to pawl tooth) increases the mechanical advantage. Another way of looking at it is that a force is applied at the handle through a distance C in order move the rack forward by a distance D. Since C is much greater than D, there is a mechanical advantage. The exact ratios to use in practice would be determined by the amount of advantage required. For example, if a 2:1 advantage is desired, it would make sense to have A be greater than or equal to 2×B. The example shown here has approximately a 6:1 mechanical advantage in the handle/pawl components of torque multiplying mechanism 130.

One of ordinary skill in the art will appreciate the various methods of providing a driver having any combination of features described herein, and of operating a combination including a driver, a torque multiplier, and a torque limiter using one or two hands as discussed herein. All such methods are contemplated as part of this disclosure even if express method-style language is not used above.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in other embodiments disclosed herein, even if such a combination of elements or features is not otherwise specifically shown or described. The same may also be varied in many ways. Such variations are not to be regarded as a departure from the disclosure, and all such modifications are intended to be included within the scope of the disclosure.

1. A method of providing a torque multiplier, limiter, and counter-torque combination for use in spinal fixation surgery comprising:
providing a drive train comprising a torque multiplier assembly and a torque limiter device, wherein an output of the torque multiplying assembly is drivingly coupled to an input of the torque limiter device;
configuring an input of the drive train to be driven by a driver handle and an output of the drive train to rotate a driver;
coupling a counter-torque connector that is configured to engage at least one member of a spinal fixation construct with a counter-torque handle to prevent rotation of the counter-torque connector and the spinal fixation construct;
providing a coupling between a component of the torque multiplier assembly and the counter-torque handle to prevent a rotation of the component;
configuring the driver handle and counter-torque handle to be simultaneously engaged by a single user, wherein torque input by the single user through the driving handle is simultaneously multiplied by the torque multiplier assembly and limited by the torque limiter device, while the single user simultaneously prevents the at least one member of the spinal fixation construct from rotating through the counter-torque handle.

2. The method of providing a torque multiplier, limiter, and counter-torque combination of claim 1, further comprising providing an axial slip coupling configured to allow axial movement of a member of the torque limiting device relative to an adjacent axially-fixed member of the drive train.

3. The method of providing a torque multiplier, limiter, and counter-torque combination of claim 1, wherein providing the coupling comprises providing a releasable coupling between the component of the torque multiplier assembly and the counter-torque handle.

4. The method of providing a torque multiplier, limiter, and counter-torque combination of claim 3, wherein providing the releasable coupling comprises providing a housing member of the torque multiplier assembly with an engagement member of the releasable coupling, and providing a cooperating engagement member of the releasable coupling on a member coupled to both the counter-torque handle and the counter-torque connector.

5.-7. (canceled)

8. The method of providing a torque multiplier, limiter, and counter-torque combination of claim 4, wherein providing the cooperating engagement member of the releasable coupling on a member coupled to both the counter-torque handle and the counter-torque connector, comprises providing the cooperating engagement member on a tubular member of the counter-torque device, which tubular member is configured to receive the housing member.

9. The method of providing a torque multiplier, limiter, and counter-torque combination of claim 4, wherein providing the engagement member comprises providing one of at least one elongated recess and at least one elongated protrusion, and wherein providing the cooperating engagement member comprises providing an other of the at least one elongated recess and the at least one elongated protrusion.

10. The method of providing a torque multiplier, limiter, and counter-torque combination of claim 9, wherein providing the cooperating engagement member of the releasable coupling on a member coupled to both the counter-torque handle and the counter-torque connector, comprises providing the cooperating engagement member on a tubular member of the counter-torque device, which tubular member is configured to receive the housing member.

11. The method of providing a torque multiplier, limiter, and counter-torque combination of claim 1, wherein configuring the driver handle and counter-torque handle to be simultaneously engaged by a single user comprises configuring both the driving handle and the counter-torque handle to extend radially from a central axis of the drive train to be simultaneously graspable by a single user, and configuring the driving handle to be rotatable relative to the counter-torque handle about the central axis.

12. The method of providing a torque multiplier, limiter, and counter-torque combination of claim 1, wherein configuring the driver handle and counter-torque handle to be simultaneously engaged by a single user comprises configuring both the driving handle and the counter-torque handle to extend radially from a central axis of the drive train to be simultaneously graspable by a single user, and configuring the driving handle to be rotatable relative to the counter-torque handle along a plane that is coincident with the central axis.

13. The method of providing a torque multiplier, limiter, and counter-torque combination of claim 12, wherein providing a drive train comprises providing the torque multiplier assembly with a linear to rotary motion conversion mechanism.

14. A torque multiplier, limiter, and counter-torque combination for use in spinal fixation surgery comprising:
a drive train comprising a torque multiplier assembly and a torque limiter device, wherein an output of the torque multiplying assembly is drivingly coupled to an input of the torque limiter device, and wherein an input of the drive train is coupled to a drive handle and an output of the drive train is coupled to a driver;
a counter-torque device comprising a counter-torque connector configured to engage at least one member of a spinal fixation construct and a counter-torque handle to prevent rotation of the counter-torque connector and the spinal fixation construct;
wherein a component of the torque multiplier assembly is coupled to the counter-torque handle to prevent a rotation of the component;
wherein a torque input through the driving handle is simultaneously multiplied by the torque multiplier assembly and limited by the torque limiter device at the driver, and wherein a counter-torque simultaneously input through the counter-torque handle simultaneously prevents rotation of the component of the torque multiplier assembly and rotation of the at least one member of the spinal fixation construct.

15. The torque multiplier, limiter, and counter-torque combination of claim 14, wherein a releasable coupling couples the component of the torque multiplier assembly to the counter-torque handle.

16. The torque multiplier, limiter, and counter-torque combination of claim 15, wherein the component comprises a housing member of the torque multiplier assembly, and the housing member comprises an engagement member of the releasable coupling, and wherein the counter-torque device comprises a cooperating engagement member of the releasable coupling.

17. The torque multiplier, limiter, and counter-torque combination of claim 16, wherein the housing member is received within a tubular member of the counter-torque device when the engagement member of the releasable coupling is coupled to the cooperating engagement member of the releasable coupling.

18. The torque multiplier, limiter, and counter-torque combination of claim 16, wherein one of the engagement member and the cooperating engagement member comprises at least one elongated recess and an other of the engagement member and the cooperating engagement member comprises at least one cooperating elongated protrusion.

19. The torque multiplier, limiter, and counter-torque combination of claim 18, wherein the housing member is received within a tubular member of the counter-torque device when the engagement member of the releasable coupling is coupled to the cooperating engagement member of the releasable coupling.

20. The torque multiplier, limiter, and counter-torque combination of claim 14, wherein the driving handle and the counter-torque handle both extend radially from a central axis of the drive train and simultaneously graspable by a single user, and wherein the driving handle is rotatable relative to the counter-torque handle about the central axis.

21. The torque multiplier, limiter, and counter-torque combination of claim 14, wherein the driving handle and the counter-torque handle both extend radially from a central axis of the drive train and simultaneously graspable by a single user, and wherein the driving handle is rotatable relative to the counter-torque handle along a plane that is coincident with the central axis.

22. The torque multiplier, limiter, and counter-torque combination of claim 21, wherein the torque multiplier assembly comprises a linear to rotary motion conversion mechanism.

23. The torque multiplier, limiter, and counter-torque combination of claim 22, wherein the linear to rotary motion conversion mechanism comprises a linear rack and pinion engaging a rotatable barrel cam.
